(19) United States
(12) Patent Application Publication
(10) Pub. No.: US 2007/0072223 A1
(43) Pub. Date: Mar. 29, 2007

(54) Compositions and methods for purifying nucleic acids

(75) Inventor: Vladimir I. Slepnev, Newton, MA (US)

Correspondence Address: PALMER & DODGE, LLP;KATHLEEN M. WILLIAMS, 111 HUNTINGTON AVENUE, BOSTON, MA, 02199, US

(73) Assignee: Primera BioSystems, Inc., Mansfield, MA (US)

(21) Appl. No.: 11/523,255

(22) Filed: Sep. 18, 2006

Related U.S. Application Data
(60) Provisional application No. 60/717,593, filed on Sep. 16, 2005.

(51) Int. Cl. C12Q 1/68; C12P 19/34; C12M 3/00

(52) U.S. Cl. 435/6; 435/91.2; 435/287.2

(57) ABSTRACT

Described herein are compositions and methods for enriching a nucleic acid sample for target sequence(s). The compositions comprise an oligonucleotide attached to a solid support, directly or indirectly, wherein the oligonucleotide does not serve as a primer for a polymerase enzyme.

This application claims the priority of U.S. provisional application Ser. No. 60/717,593 filed Sep. 16, 2005, the entirety of which is incorporated herein by reference.

### FIELD OF THE INVENTION

The invention relates in general to compositions and methods of enriching a target nucleic acid, as well as methods to perform a polymerase reaction.

### BACKGROUND OF THE INVENTION

The ability to isolate nucleic acids from a biological sample is often requisite to a vast array of molecular biological methods and protocols, as well as in a growing number of downstream diagnostic and biotechnological uses. For example, the isolation of ribonucleic acids (RNA) is the starting point for the analysis of gene expression. Likewise, isolation of deoxyribonucleic acids (DNA) is the starting point for determining the presence of mutations, alleles, or polymorphisms within genes to determine, for example, a subject's predisposition towards disease.

Numerous approaches to the isolation of nucleic acids are known in the art, including the use of organic solvents, chromatography fractionation, and density gradient centrifugation. Messenger RNA (mRNA), for example, is often purified by chromatography using oligo-dT oligonucleotides attached to solid support, by exploiting the high binding affinity between oligo d(T) and poly-A tails of mRNAs. In other instances, magnetic beads have been used to isolate and purify mRNA, to construct solid-phase cDNA libraries, and for PCR, differential display and subtractive hybridization applications. For example, WO 96/09313 describes the isolation of mRNA using oligo-dT oligonucleotides attached to magnetic beads.

Locked nucleic acids (LNAs) are recently described analogs of natural nucleic acids exhibiting certain ideal properties, for example, in having an unusually high melting temperature in hybrids formed with DNA or RNA (Koshkin et al. (1998) Tetrahedron, 54:3607-3630). Methods of isolating nucleic acids using such LNAs have been described, for example in U.S. Pat. No. 6,303,315 and U.S. patent application Ser. No. 10/601,140.

### SUMMARY OF THE INVENTION

The present invention provides compositions comprising an oligonucleotide attached to a solid support. The present invention also provides a method of enriching a target nucleic acid using such a composition. Also provided is a method of performing a polymerase reaction using the compositions provided herein.

In a first aspect of the present invention, a composition is provided which comprises a first oligonucleotide which is attached to a solid support, wherein the oligonucleotide does not serve as a primer for synthesis by a polymerase enzyme. In one embodiment, the oligonucleotide is attached at its 3′ end to the solid support, thereby preventing it from serving as a primer for synthesis. In another embodiment, the solid support is magnetic. In yet another embodiment, the oligonucleotide comprises at least one non-natural nucleic acid. One example of such a non-natural nucleic acid is a locked nucleic acid (LNA).

The composition of the present invention can further comprise a second oligonucleotide. The second oligonucleotide comprises a tag binding sequence is substantially complementary to and hybridizes to a tag sequence within the first oligonucleotide. In one embodiment, the second oligonucleotide further comprises a target binding sequence, which is covalently linked to the tag binding sequence. The tag binding sequence and the target binding sequence of the second oligonucleotide can be attached directly or coupled via a linker. In one embodiment, the target binding sequence is attached to the 3′ end of the tag binding sequence. The linker can be a nucleic acid (natural or non-natural), or can be a chemical spacer. In one embodiment, the linker can be cleavable.

The invention further provides a method of enriching a target nucleic acid in a sample. The method comprises the steps of providing a sample, contacting the sample with the composition of the invention under conditions that allow the target nucleic acid and the composition to form a hybrid complex, and separating the hybrid complex, whereby the target nucleic acid is enriched. The method can employ a composition comprising either one or two oligonucleotides.

Finally, the invention provides a method of performing a polymerase reaction. The method comprises the steps of providing a sample containing nucleic acids, contacting the sample with the composition of the present invention under conditions that allow a hybrid complex to form between the target nucleic acid and the composition, and extending the hybrid complex using a polymerase. The method employs a composition comprising two oligonucleotides, wherein the second nucleotide contains a target binding sequence. The method can further comprise purifying the polymerase reaction product.

Definitions.

As used herein, “enriching” a nucleic acid refers to the process of significantly increasing the concentration of a given nucleic acid relative to the concentration of at least one other nucleic acid present in a sample. As used herein, “enriching” a nucleic acid refers to an increase of at least 2-fold, for example, 3-fold, 5-fold, 10-fold, 30-fold, 100-fold, 300-fold, 1000-fold or more, of a target nucleic acid from a sample.

As used herein, the term “sample” refers to a biological material which is isolated from its natural environment and containing a polynucleotide. A “sample” according to the invention may consist of purified or isolated polynucleotide, or it may comprise a biological sample such as a tissue sample, a biological fluid sample, or a cell sample comprising a polynucleotide. A biological fluid includes blood, plasma, sputum, urine, cerebrospinal fluid, ravages, and leukophoresis samples. A sample of the present invention can comprise any plant, animal, bacterial or viral material containing a polynucleotide.

As used herein, a “target binding sequence” refers to a sequence which hybridizes to a target nucleic acid. As used herein, the terms “target polynucleotide” and “target nucleic acid” refer to a polynucleotide to be enriched from a sample. A “target nucleic acid” of the present invention contains a known sequence of at least 20 nucleotides, preferably at least 50 nucleotides, more preferably at least 100 or more nucleotides, for example, 500 or more nucleotides. A “target nucleic acid” of the invention may be a naturally occurring polynucleotide (i.e., one existing in nature without human intervention), or a recombinant polynucleotide (i.e., one existing only with human intervention), including but not limited to genomic DNA, cDNA, plasmid DNA, total RNA, mRNA, tRNA, and rRNA. The target polynucleotide also includes amplified products of itself, for example, as in a polymerase chain reaction. According to the invention, a “target polynucleotide” or “target nucleic acid” may contain a modified nucleotide which includes phosphorothioate, phosphite, ring atom modified derivatives, and the like. According to the invention, a sample may contain one or more nucleic acids that are target nucleic acids. For example, where a “target binding sequence” comprises a sequence found on several nucleic acids, such a target binding sequence will have several target nucleic acids (e.g., for a target binding sequence of “TTTTTTTT” (SEQ ID NO: 1), the “target nucleic acid” can be any transcript containing polyadenylation, or an internal poly-A sequence).

As used herein, the term “oligonucleotide” refers to a short polynucleotide, typically less than or equal to 150 nucleotides long (e.g., between 5 and 150, between 10 to 100, or between 15 to 50 nucleotides in length). However, as used herein, the term is also intended to encompass longer or shorter polynucleotide chains. An “oligonucleotide” can hybridize to other polynucleotides. An oligonucleotide has a “5′-terminus” and a “3′-terminus” because polynucleotide phosphodiester linkages occur to the 5′ carbon and 3′ carbon of the pentose ring of the substituent mononucleotides. The end of an oligonucleotide at which a new linkage would be to a 5′ carbon is its 5′ terminal nucleotide. The end of an oligonucleotide at which a new linkage would be to a 3′ carbon is its 3′ terminal nucleotide. A terminal nucleotide, as used herein, is the nucleotide at the end position of the 3′- or 5′-terminus. As used herein, a polynucleotide sequence, even if internal to a larger polynucleotide (e.g., a sequence region within a polynucleotide), also can be said to have 5′- and 3′-ends.

By “locked nucleic acid” or “LNA” is meant a locked nucleic acid, which is a nucleotide containing a methylene bridge that connects the 2′-oxygen of ribose with the 4′-carbon of ribose, and is described, for example, in WO 99/14226.

As used herein, the term “complementary” refers to the concept of sequence complementarity between regions of two polynucleotide strands or between two regions of the same polynucleotide strand. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Fully complementary” refers to a first polynucleotide that is 100% or “fully” complementary to a second polynucleotide and thus forms a base pair at every nucleotide position. “Substantially complementary” refers to a first polynucleotide that is not 100% complementary (e.g., 90%, or 80% or 70% complementary) contains mismatched nucleotides at one or more nucleotide positions (e.g., one, two, three, or four mismatches). In one embodiment, two complementary polynucleotides are capable of hybridizing to each other under high stringency hybridization conditions. For example, for membrane hybridization (e.g., Northern hybridization), high stringency hybridization conditions are defined as incubation with a radiolabeled probe in 5×SSC, 5× Denhardt's solution, 1% SDS at 65° C. Stringent washes for membrane hybridization are performed as follows: the membrane is washed at room temperature in 2×SSC/0.1% SDS and at 65° C. in 0.2×SSC/0.1% SDS, 10 minutes per wash, and exposed to film. For hybridization of nucleic acids immobilized on solid support, hybridization can be performed under conditions similar to those employed for the polymerase chain reaction, as is well known in the art. For example, hybridization can be performed under high stringency by annealing at a temperature of at least 50° C. in the presence of 1×PCR buffer. As used herein, a “hybrid complex” refers to a complex formed between two complementary strands of nucleic acids.

As used herein, the terms “polymerase enzyme”, “nucleic acid polymerase” and “polymerase” are interchangeable terms which refer to an enzyme that catalyzes the polymerization of nucleoside triphosphates, and can include DNA polymerases, RNA polymerases, and reverse transcriptases. Generally, the enzyme will initiate synthesis at the 3′-end of the primer annealed to the target sequence, and will proceed in the 5′-direction along the template, until synthesis terminates. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase. Similarly, commonly used RNA polymerases include, but are not limited to, the T7 RNA polymerase, T3 RNA polymerase, and SP6 RNA polymerase. Finally, reverse transcriptases include, for example, the AMV reverse transcriptase, M-MuLV reverse transcriptase, PowerScript® (BD Biosciences, Palo Alto, Calif.) StrataScript® and AccuScript® reverse transcriptases (Stratagene, La Jolla, Calif.), and SuperScript® (Invitrogen, Carlsbad, Calif.). A “polymerase reaction”, as used herein, encompasses all polymerization reactions by polymerase enzymes.

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

As used herein, “Tₘ” and “melting temperature” are interchangeable terms which are the temperature at which 50% of a population of double-stranded polynucleotide molecules becomes dissociated into single strands.

A “cleavable linker” as used herein, refers to any structure which acts to link structures and which can be specifically and conveniently cleaved. As used herein, a “cleavable linker” can be a linker nucleotide sequence containing a restriction enzyme recognition sequence. A “cleavable linker,” as used herein, can also include a structure which is more susceptible to nuclease cleavage (for example, a ribonucleic acid sequence within an otherwise deoxyribonucleic acid oligonucleotide), such that the structure can be cleaved by addition of an enzyme with little or no sequence specificity (e.g., a ribonuclease). Finally, a “cleavable linker” can also include a chemical linker or adaptor which can be cleaved using reagents, for example, reducing agents.

As used herein, the terms “magnetic particle” and “paramagnetic particle” or “superparamagnetic particle,” are interchangeable terms which refer to particles or beads which are magnetically responsive, i.e., a particle that is attracted by a magnetic field. Generally, to form paramagnetic or superparamagnetic particles, metal oxide particles are coated with polymers that are relatively stable in water. As used herein, the term “metal particle” refers to any oxide of a metal or metal alloy having paramagnetic or superparamagnetic properties.

### BRIEF DESCRIPTION OF THE DRAWINGS

FIG. 1 shows two examples of one oligonucleotide composition. A. Single oligonucleotide (SEQ ID NO: 2) attached at its 3′ end to a magnetic bead. B. Single oligonucleotide (SEQ ID NO: 2) attached at an internal location to a silica bead; the 3′ terminal nucleotide is a substituted dideoxy nucleotide which prevents the oligonucleotide from serving as a primer for a polymerase enzyme. Both examples show attachment of oligonucleotides via an optional spacer.

FIG. 2 shows a one oligonucleotide composition comprising a latex bead, attached via an optional spacer to a first oligonucleotide consisting of a target binding sequence (SEQ ID NO 3) composed of both DNA and LNA.

FIG. 3 shows an example of a two oligonucleotide composition. The first oligonucleotide (SEQ ID NO 2) is covalently attached at its 3′ OH moiety to a magnetic bead via an optional spacer. The second oligonucleotide (SEQ ID NO: 4) consists of a tag binding sequence (to form a hybrid with the first oligonucleotide) as well as a target binding sequence (to hybridize to a target nucleic acid).

FIG. 4 shows a method of enriching for a target nucleic acid using a two oligonucleotide composition. After forming a hybrid complex with the target nucleic acid as shown in FIG. 4A, the complex is separated, optionally washed, and the target nucleic acid is dissociated (FIG. 4B) in this case by applying a temperature higher than the melting temperature of the target:oligonucleotide hybrid complex but lower than the first:second oligonucleotide complex.

FIG. 5 shows another method for enriching a target nucleic acid using a two oligonucleotide composition. After forming a hybrid complex with the target nucleic acid (FIG. 5A), the complex is separated, optionally washed, and the enriched target nucleic acid is dissociated (FIG. 5B), in this case by cleavage of the optional cleavable linker. The enriched target nucleic acid is dissociated still complexed with the target binding sequence of the second oligonucleotide, which can serve as a primer for a template-dependent polymerase reaction.

FIG. 6 illustrates the use of a “universal” first oligonucleotide composition, with which numerous second oligonucleotides can be used. The second oligonucleotides shown share a common target binding sequence to form a hybrid complex with the tag within the first oligonucleotide, but contain distinct target binding sequences that hybridize to different targets.

FIG. 7 illustrates that multiple target binding sequences can be attached to a single bead using at least two methods. A. First oligonucleotide containing target binding sequence “A” is attached to a bead. B. A second oligonucleotide containing target binding sequence “A” hybridizes to first oligonucleotide, which in turn is attached to a bead.

FIG. 8 illustrates two distinct means to enrich for multiple target nucleic acids. A. Attachment of first oligonucleotides with distinct target binding sequences (A-D) on a single bead. B. Attachment of second oligonucleotides with distinct target binding sequences (A-D), for example as shown in FIG. 6, hybridized through a common tag binding sequence to first oligonucleotides attached to the bead.

FIG. 9 illustrates other means of enriching for diversity. A. Distinct beads, each with a different target binding sequence. B. Each bead has oligonucleotides which hybridize to distinct target binding sequences.

### DETAILED DESCRIPTION

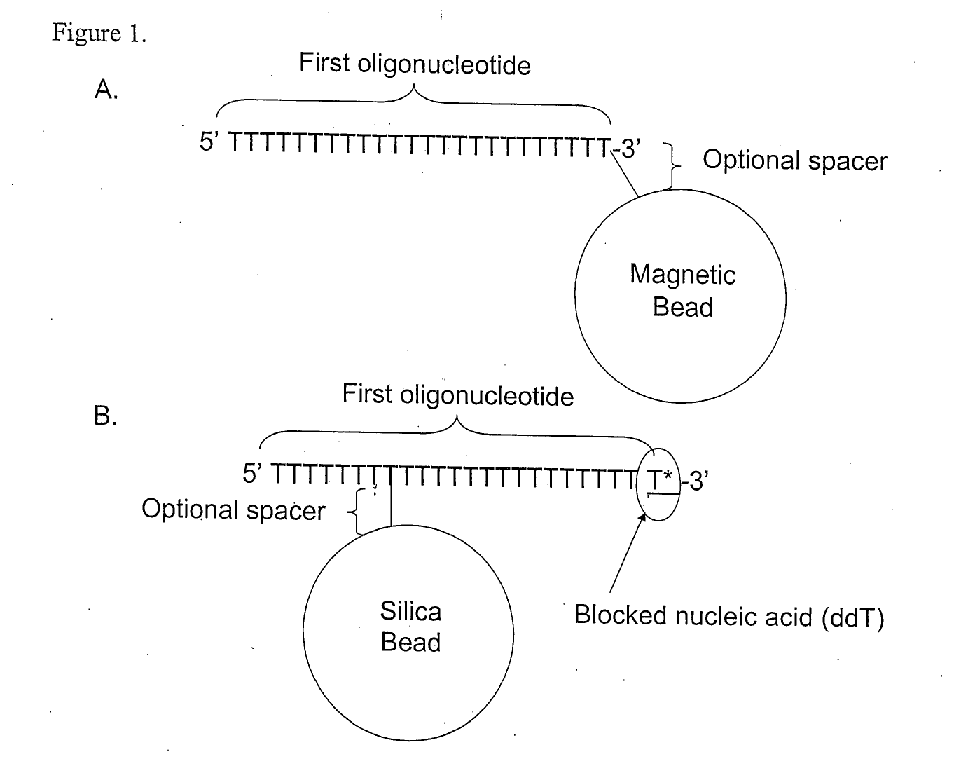

The compositions described herein comprise, in its simplest form, a first oligonucleotide attached to a solid support. The first oligonucleotide does not serve as a primer for a polymerase enzyme. An example of such a nucleic acid/solid support composition is shown in FIG. 1. The first oligonucleotide can be attached to the solid support via an optional spacer (See, for example, FIG. 1A. In one embodiment, the first oligonucleotide is attached at its 3′ end to a solid support (e.g., a magnetic bead). The solid support can also be attached to the first oligonucleotide at internal positions, as is exemplified in FIG. 1B. The first oligonucleotide can be either covalently or non-covalently attached to the solid support. In a preferred embodiment, the first oligonucleotide is covalently attached to the solid support, either directly or through a spacer molecule.

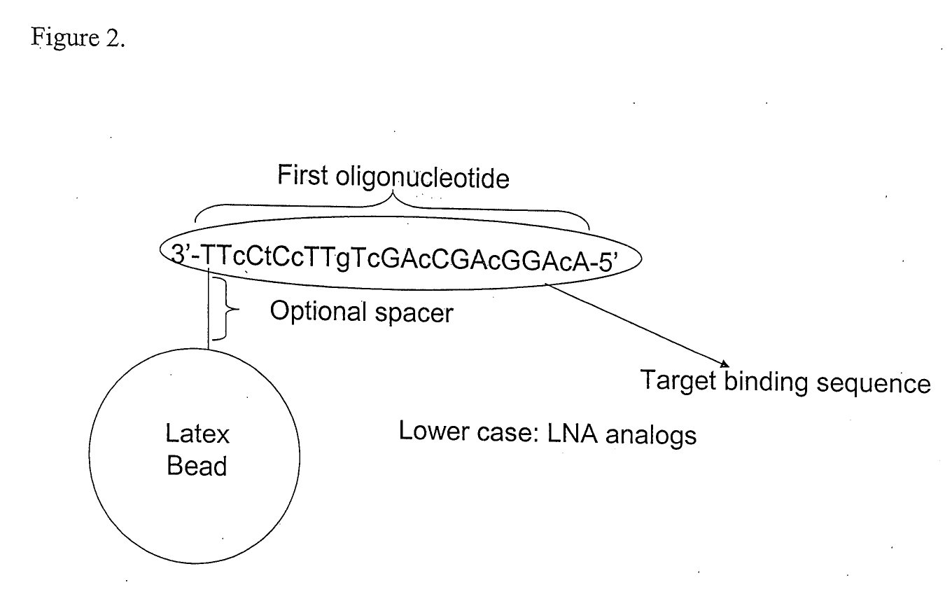

The first oligonucleotide can comprise a target binding sequence, which is at least substantially complementary (i.e., substantially or fully complementary) to a portion of a target nucleic acid (See, for example, FIG. 2). The first oligonucleotide can comprise natural, non-natural nucleic acids, or combinations thereof. In one embodiment, the first oligonucleotide comprises at least one locked nucleic acid (See, for example, FIG. 2).

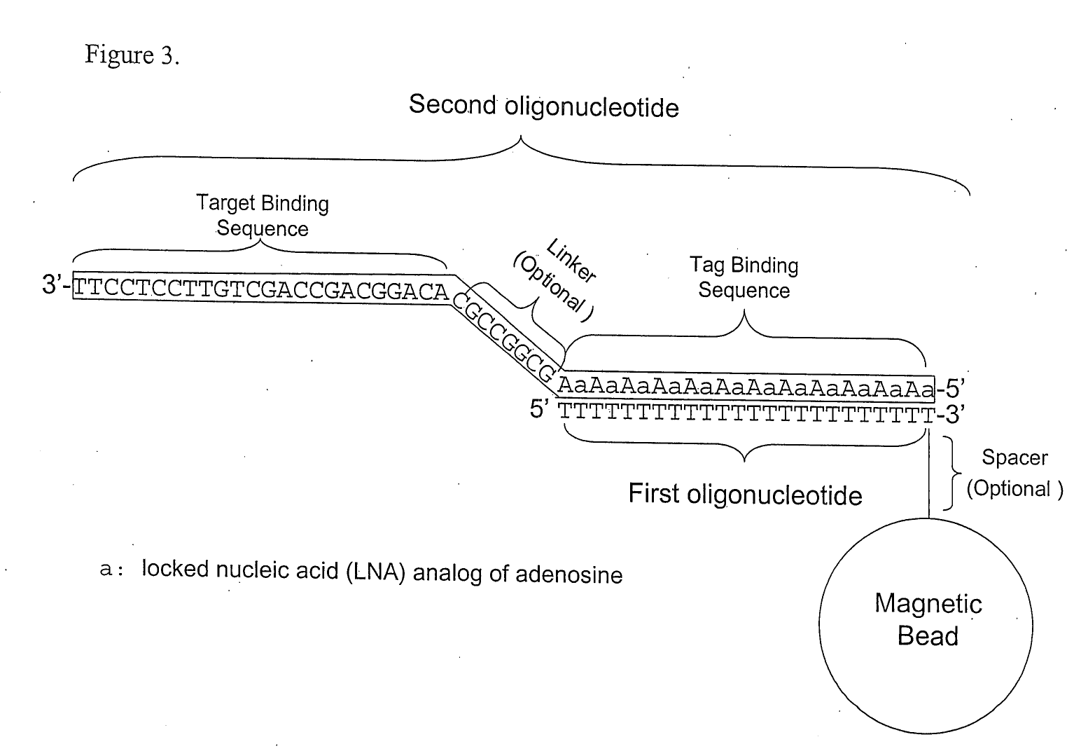

The composition can further comprise a second oligonucleotide (See, for example, FIG. 3). The second oligonucleotide comprises a tag binding sequence which is complementary forms a hybrid complex with a tag within the first oligonucleotide. The second oligonucleotide can further comprise a target binding sequence (FIG. 3). The target binding sequence can be located at any position relative to the tag binding sequence. In one embodiment, the target binding sequence is located 3′ of the tag binding sequence (See, for example, FIG. 3). The target binding sequence and tag binding sequence can additionally be separated by an optional linker, which can be a natural (as is shown in FIG. 3), non-natural nucleic acid, a combination thereof, or a chemical linker. The linker can be a cleavable linker.

In one embodiment, the first oligonucleotide and/or second oligonucleotide comprise a DNA homopolymer. In another embodiment, the first oligonucleotide and/or second oligonucleotide comprise a RNA homopolymer. In still another embodiment, the first oligonucleotide and/or second oligonucleotide comprise a LNA homopolymer. In yet another embodiment, the first oligonucleotide and/or second oligonucleotide comprise a DNA/LNA copolymer. In another embodiment, the first oligonucleotide and/or second oligonucleotide comprise a RNA/LNA copolymer.

Different portions within the first oligonucleotide and/or second oligonucleotide can comprise different nucleic acid compositions. In one embodiment, the tag of the first oligonucleotide comprises at least one LNA. In another embodiment, the tag binding sequence of the second oligonucleotide comprises at least one LNA.

As previously described, the target binding sequence, whether on the first oligonucleotide or second oligonucleotide, forms a hybrid complex with a portion of a target nucleic acid. It is at least substantially complementary to a part of the target nucleic acid. The target binding sequence is typically between 5 and 50 nucleotides in length, for example between 5 and 45, between 5 and 40, or between 7 and 35 nucleotides in length.

The hybrid complex formed between the first and second oligonucleotide (i.e., the complex between the tag:tag binding sequence) can have a higher melting temperature than the melting temperature of the hybrid complex formed between the target nucleic acid and the target binding sequence. In one embodiment, the melting temperature of the tag:tag binding sequence hybrid complex is at least 5° C. higher than that of the target nucleic acid:target binding sequence.

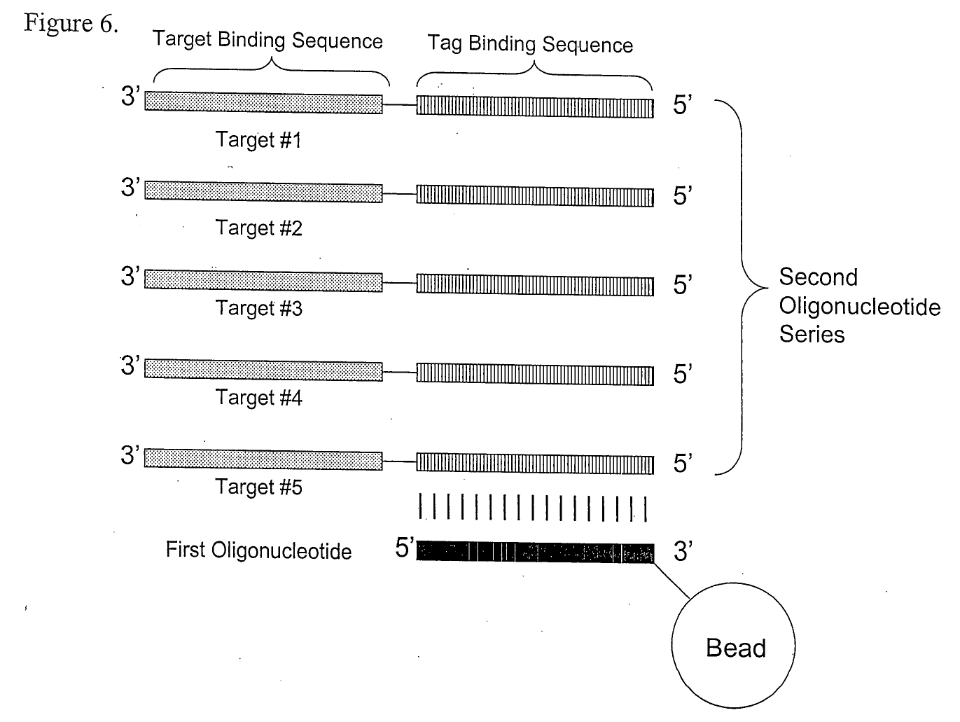

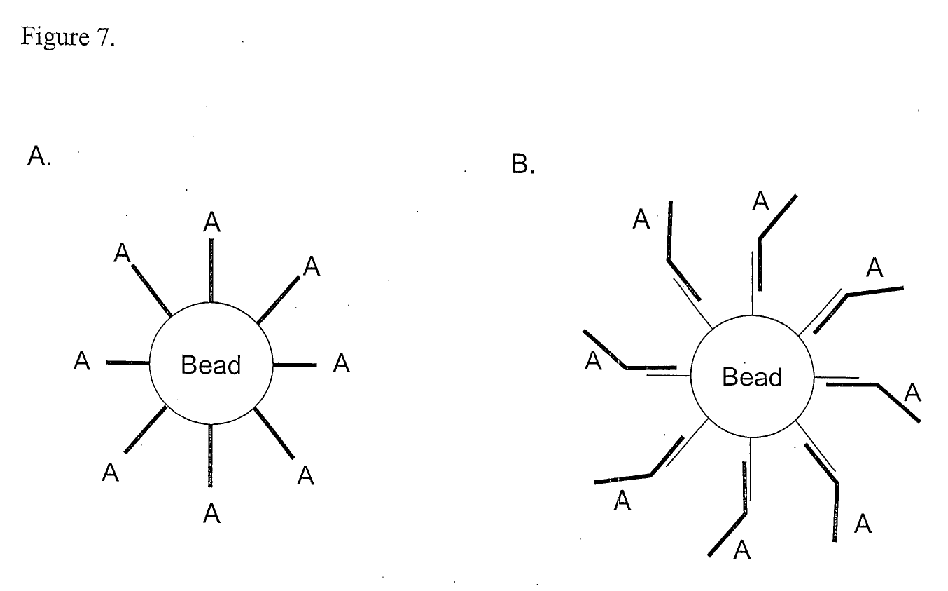

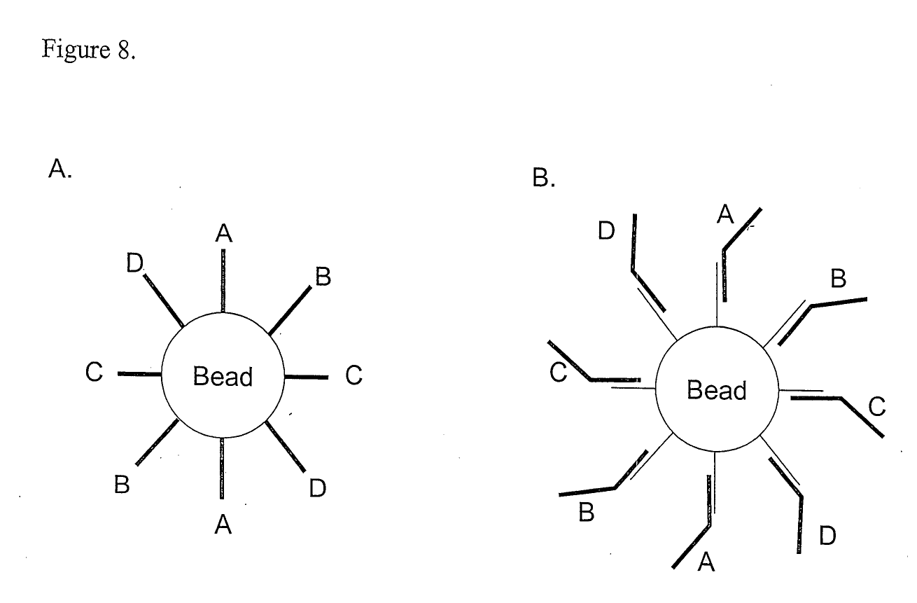

The compositions described herein also encompass compositions comprising target binding sequences for multiple target nucleic acids. Multiple copies of a target binding sequence to the same target nucleic acid can be present on a single solid support (See, for example, FIGS. 6-8), either by attaching multiple copies of a first oligonucleotide to a single support (FIG. 7A), or by hybrid complex formation with second oligonucleotides, all containing the same target binding sequences (FIG. 7B). In addition, target bindings sites to a plurality of different target nucleic acids can also be present on a single solid support (e.g., a bead), as is shown in FIG. 8, using only first oligonucleotides containing different target binding sequences (FIG. 8A), or by complex formation to second oligonucleotides containing different target binding sequences (FIG. 8B). Second oligonucleotides with different target binding sequences but with common tag binding sequences can be hybridized to first oligonucleotides, all attached to a support and all containing identical tags (FIG. 6).

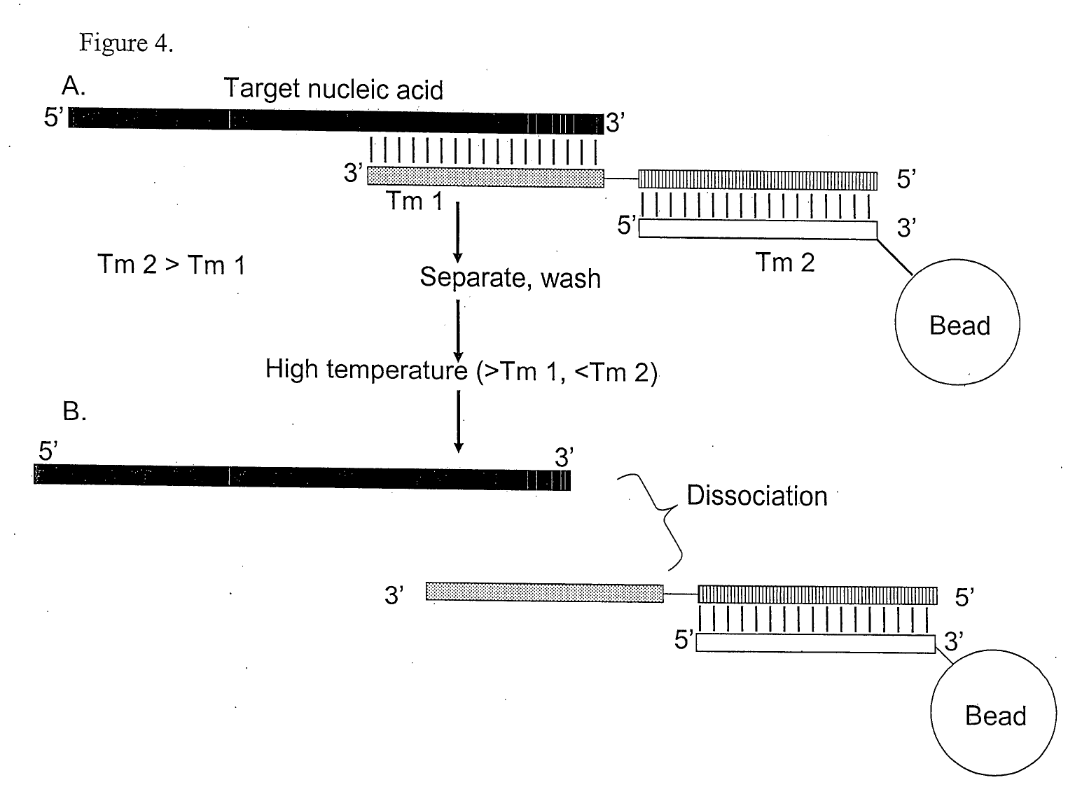

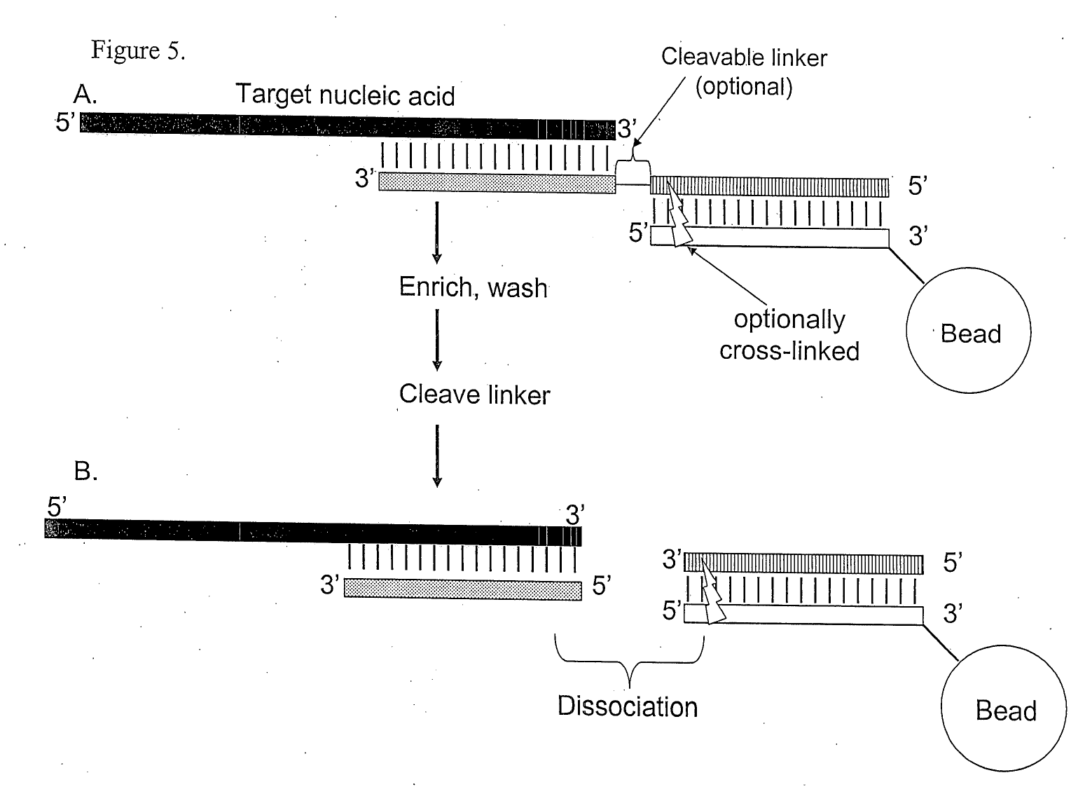

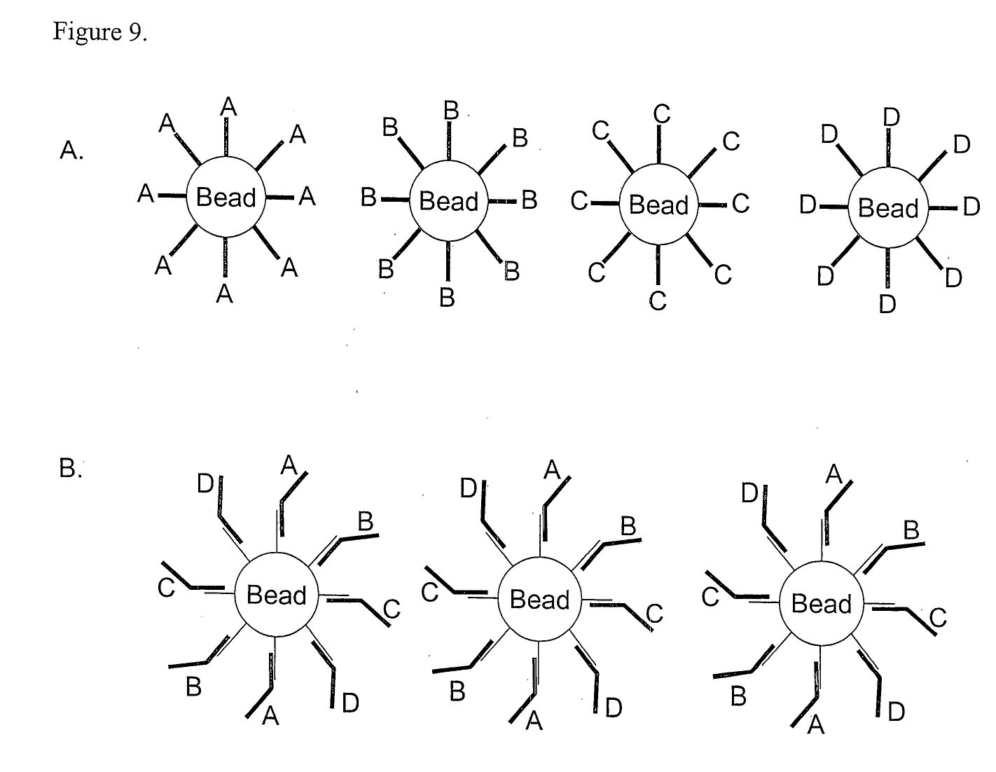

Also disclosed are methods of enriching for a target nucleic acid using the nucleic acid/solid support compositions of the invention. The method comprises the steps of providing a sample containing nucleic acid; contacting the sample with a nucleic acid/solid support composition described herein under conditions that allow the target nucleic acid and composition to form a hybrid complex (See, for example, FIGS. 4A & 5A); and, separating the hybrid complex from the rest of the sample. In one embodiment, the method further comprises the step of dissociating the hybrid complex after separating the hybrid complex (e.g., FIG. 4B and FIG. 5B). The method also contemplates the enrichment of a plurality of different target nucleic acids, using either a solid support with target binding sequences to the different targets (FIG. 8), or by mixing solid supports, each of which has a single target binding sequence (See, for example, FIG. 9A and FIG. 9B).

Finally, the invention provides a method of performing a polymerase reaction. The method comprises the steps of providing a sample containing nucleic acids, contacting the sample with the composition of the present invention under conditions that allow a hybrid complex to form between the target nucleic acid and the composition, and extending the hybrid complex using a polymerase. The method employs a composition comprising two oligonucleotides, wherein the second nucleotide contains a target binding sequence. The method can further comprise purifying the polymerase reaction product.

Nucleotides

According to the present invention, the oligonucleotide can comprise natural, non-natural nucleotides and analogs. The oligonucleotide may be a nucleic acid analog or chimera comprising nucleic acid and nucleic acid analog monomer units, such as 2-aminoethylglycine, a peptide nucleic acid (PNA), or a locked nucleic acid (LNA). For example, part or all of the oligonucleotide may be LNA or a LNA/nucleic acid (DNA or RNA) chimera.

In one embodiment, the oligonucleotide comprises at least one locked nucleic acid. Locked nucleic acids represent a class of conformationally restricted nucleotide analogues described, for example, in WO 99/14226, which hybridize more strongly to both DNA and RNA than naturally occurring nucleotides. Such a locked nucleotide contains a methylene bridge that connects the 2′-oxygen of ribose with the 4′-carbon of ribose:

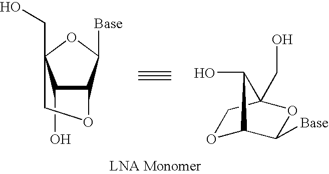

Oligonucleotides containing the locked nucleotide are described in Koshkin, A. A., et al., Tetrahedron (1998), 54: 3607-3630) and Obika, S. et al., Tetrahedron Lett. (1998), 39: 5401-5404), both of which are incorporated herein by reference in their entirety. Introduction of a locked nucleotide into an oligonucleotide improves the affinity for complementary sequences and increases the melting temperature by several degrees (Braasch, D. A. and D. R. Corey, Chem. Biol. (2001), 8:1-7). The compositions and methods described herein can include any of the LNAs known in the art, for example, those disclosed in WO 99/14226 and in Latorra D, et al., 2003. Hum. Mutat. 22: 79-85, both of which are incorporated herein by reference. More specific binding can be obtained and more stringent washing conditions can be employed using LNA analogs, with the advantage that the amount of background noise is reduced significantly.

In one embodiment, the first oligonucleotide comprises at least one locked nucleic acid. In another embodiment, the second oligonucleotide comprises at least one locked nucleic acid. In still another embodiment, the first and/or second oligonucleotide comprises alternating locked and natural nucleic acids for at least a portion of its sequence. For example, the second oligonucleotide can contain:

| | |
|---|---|
| 5′-AaAaAaAaA-3′ | (SEQ ID NO: 5) |

within the tag binding sequence, wherein “A” denotes an adenosine, and “a” denotes a locked nucleic acid analog thereof.
First Oligonucleotide

The composition of the present invention comprises, in its simplest form, a first oligonucleotide, attached to a solid support. The first oligonucleotide does not serve as a primer of a polymerase enzyme.

The composition of the present invention encompasses any situation in which the first oligonucleotide can not serve as a primer for synthesis by a polymerase enzyme. In one embodiment, the 3′-end of the first oligonucleotide (e.g., the 3′ OH moiety) is blocked from serving as a polymerase substrate by attachment of a solid support, either directly or via a spacer molecule. In another embodiment, the 3′ nucleotide is modified so that it cannot serve as a primer for further synthesis by a polymerase (e.g., a dideoxy nucleotide). In yet another embodiment, the 3′ OH moiety of the 3′-end nucleotide can be attached to another molecule, for example a fluorophore or any other moiety that can prevent the addition of another nucleotide by a polymerase. Likewise, modification of an adjacent or nearby nucleotide, for example by attachment of a large molecule, to prevent access by the polymerase by steric hindrance, can also be a means, within the scope of the present invention, to prevent the first oligonucleotide from serving as a polymerase primer.

The sequence of the first oligonucleotide depends upon its use, the target nucleic acid sequence, and whether a second oligonucleotide is used. In embodiments in which only the first oligonucleotide is used, the first nucleotide contains a target binding sequence. In embodiments in which a second oligonucleotide is also used, the first oligonucleotide contains a tag sequence that is at least substantially complementary (i.e., either substantially complementary or fully complementary) to the tag binding sequence of the second oligonucleotide. Therefore, in one embodiment, the first oligonucleotide contains a tag sequence that is at least substantially complementary (i.e., either substantially complementary or fully complementary) to the tag binding sequence of the second oligonucleotide. In another embodiment, the first oligonucleotide contains sequence that is at least substantially complementary (i.e., either substantially complementary or fully complementary) to a portion of the target nucleic acid. In yet another embodiment, the first and second oligonucleotides can both contain a target binding sequence (identical, overlapping or different). The target binding sequence can be complementary to a target nucleic acid located at the 5′ end, 3′ end or internally within the target nucleic acid sequence.

In addition to a target binding sequence and/or a tag sequence, the first oligonucleotide can contain additional sequences, including sequences which allow transcription (such as RNA polymerase binding sites), restriction endonuclease cleavage, a hairpin loop, and/or a unique identification bearing information, or “bar-coding”, such that the identity of the oligonucleotide can be easily retrieved.

The first oligonucleotide of the composition is between 5 and 150 nucleotides in length (e.g., between 6 and 100, between 7 and 50, or between 8 and 40 nucleotides in length). The length of the first oligonucleotide depends on a variety of factors, including the composition of the oligonucleotide (RNA, DNA and/or LNA) as well as that of the target nucleic acid or second oligonucleotide, whichever one it is designed to bind; the concentration of the target nucleic acid sequence or second oligonucleotide; and, the complexity of the sample, to name just a few factors. The ideal Tₘ of target binding sequence is between 35° C. and 85° C., typically between 40° C. and 80° C., for example, between 45° C. and 75° C.

Solid Support

The composition of the present invention comprises an oligonucleotide attached to a solid support. As used herein, a solid support can be one of many known in the art, including but not limited to magnetic (or paramagnetic) particles, silica-based matrices, cellulosic materials, plastic materials, membrane-based matrices and beads comprising surfaces including, but not limited to styrene, latex or silica based materials and other polymers. In one embodiment, the solid support is a bead. In another embodiment, the composition of the present invention comprises magnetic beads. Magnetic beads are produced in various ways; often paramagnetic metals, such as metal oxides, are encapsulated with a suitable coating material, such as a polymer or a silicate, to produce coated beads that are about 1 μm-100 μm in diameter. Magnetic beads useful for the methods described herein can be obtained commercially from any of several sources, including, but not limited to, Bioclone, Inc. (San Diego, Calif.), Dynal Biotech, LLC. (Brown Deer, Wis.), Chemicell GmbH (Berlin, Germany), which produce magnetic beads with numerous functional groups for simplified coupling to oligonucleotides, including amine-terminated, DADPA-terminated, carboxy-terminated, epoxy-activated, aldehyde-modified, hydrazide-modified, IDA-modified, and silica-modified beads, to name a few.

It is well known by those of skill in the art that oligonucleotides can be synthesized with certain chemical and/or capture moieties, such that they can be coupled to solid supports. Examples of attaching oligonucleotides to solid supports can be found, for example, in U.S. Patent Application No. U.S. 2003/0165912 A1, which is hereby incorporated herein in its entirety. Suitable capture moieties include, but are not limited to, biotin, a hapten, a protein, a nucleotide sequence, an antigenic moiety, or a chemically reactive moiety. Such oligonucleotides may either be used first in solution and then captured onto a solid support, or first attached to a solid support and then used in a detection reaction. In one embodiment, the oligonucleotide is attached to the support, e.g., a bead, by a chemically reactive moiety that reacts with a moiety at the 3′ end of the oligonucleotide to be coupled.

The solid support can be attached to the first oligonucleotide, either directly or through the use of a spacer. In one embodiment, the solid support is attached at the 3′ end of the oligonucleotide. As will be apparent to one of skill in the art, the solid support can be attached to the oligonucleotide directly, or via a spacer of, for example, between 1 and 40 atoms in length. Examples of suitable spacers are described in U.S. Pat. No. 5,770,716, which is hereby incorporated herein by reference. In one embodiment, the first oligonucleotide is attached at its 3′ end to the solid support, either directly or through a spacer. The spacer can be PEG-based or any other common linkage known in the art.

Second Oligonucleotide

As described previously, the compositions described herein can further comprise a second oligonucleotide. Where a second oligonucleotide is employed, the second oligonucleotide hybridizes with the first oligonucleotide. The second oligonucleotide contains a tag binding region which is at least substantially complementary to a portion of the first oligonucleotide.

As described previously for the first oligonucleotide, the second oligonucleotide can also comprise natural (e.g., ribonucleic acid and/or deoxyribonucleic acid), non-natural nucleic acids, or combinations thereof. In one embodiment, the second oligonucleotide comprises at least one locked nucleic acid (LNA). In another embodiment, the second oligonucleotide comprises at least one locked nucleic acid within the tag binding sequence.

In one embodiment, the second oligonucleotide further comprises a target binding sequence which is covalently linked to the tag binding sequence. The target binding sequence is complementary to a sequence of the target nucleic acid, either within the sequence or near or at either the 5′ or 3′ end. The length of the target binding sequence depends on several factors, including the melting temperature of the tag binding sequence hybrid, complexity of the sample, ionic conditions, and the like.

As will be discussed in further detail below, the compositions of the present invention can be used to enrich for a target nucleic acid. In general, the target binding sequence is designed such that its hybrid complex with the target nucleic acid has a sufficiently high melting temperature to allow for enrichment. In one such embodiment, the target binding sequence of the second oligonucleotide is designed such that its melting temperature is at least five degrees lower than that of the tag binding sequence, for example at least 5° C., 7° C., 10° C., 12° C., 15° C., or more. The equation for calculating the Tₘ of polynucleotides is well known in the art. For example, the Tₘ may be calculated by the following equation: Tₘ=69.3+0.41×(G+C)%−650/L, wherein L is the length of the oligonucleotide in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically. The stability and melting temperature of sequences can also be determined, for example, using programs such as mfold (Zuker (1989) Science, 244, 48-52) or Oligo 5.0 (Rychlik & Rhoads (1989) Nucleic Acids Res. 17, 8543-51). Methods for calculating the Tₘ of natural and non-natural nucleic acids are also known in the art. For example, the melting temperature LNA-DNA hybrids can be calculated using methods known in the art, for example as described in McTigue et al. (2004) Biochemistry, 43, 5388-5405, Tolstrup et al., (2003) Nucl. Acid Res. 31, 3758-62, both references of which are incorporated herein by reference in their entirety.

The target binding sequence can be linked directly to the tag binding sequence, or be separated by a linker. The linker can be a nucleic acid sequence (natural and/or non-natural) of one to 40 bases (e.g., 1 to 40, 2 to 35, 4 to 30, or 6 to 25 bases) or a chemical spacer. Furthermore, the linker can comprise a cleavable linker. In one embodiment, the cleavable linker is a rare restriction enzyme recognition sequence, either due to the fact that the recognition sequence is long (e.g., at least 6 base pairs, 7 base pairs, 8 base pairs or more), or due to its sensitivity to methylation. While the cleavable linker of the oligonucleotide as described herein may not be provided as a double stranded sequence, formation of a hybrid complex, either with the other oligonucleotide of the composition, or with the target nucleic acid, can result in a double stranded sequence which can serve as a substrate for cleavage by certain enzymes, for example, restriction endonucleases. Alternatively, the cleavable linker can contain different nucleic acids from the rest of the oligonucleotide, which can be cleaved using non-specific endonucleases. For example, an oligonucleotide consisting of deoxyribonucleic acids can contain a cleavable linker which is composed of ribonucleic acids, thereby rendering the cleavable linker sensitive to ribonucleases. Finally, the cleavable linker can be a cleavable chemical linker. Many such cleavable linkers are known in the art: non-limiting examples include disulfide cleavable linker (U.S. Pat. No. 5,412,087) and UV-labile linker (Olejnik et al., (1999) Nucleic Acids Research 27:4626-4631), incorporated herein by reference.

The first and second oligonucleotide can further be joined by crosslinkers. In one embodiment, the first oligonucleotide and/or second oligonucleotide contains a crosslinking agent. In another embodiment, the first oligonucleotide and/or second oligonucleotide contains a crosslinking agent within the tag sequence which forms a hybrid complex with a tag binding sequence of a second oligonucleotide. The crosslinking moiety can be directly incorporated into synthetic oligonucleotides at the time of synthesis through the use of appropriately modified nucleoside or nucleotide derivatives. Alternatively, the crosslinking molecules can be introduced onto the probe through photochemical or chemical monoaddition. In some cases, the crosslinking moiety may be incorporated into a nucleic acid enzymatically by using an appropriately modified nucleotide or oligonucleotide which contains a cross-linking moiety.

The crosslinking moiety which is employed on the nucleic acid composition may be any chemical moiety capable of forming a covalent crosslink between first and second oligonucleotides. For instance, the precursor to the crosslinking moiety can optionally be a coumarin, furocoumarin, or a benzodipyrone. Several of such crosslinkers useful in the present invention are known to those skilled in the art. For example, U.S. Pat. Nos. 4,599,303 and 4,826,967, also incorporated herein in their entirety disclose crosslinking compounds based on furocoumarin which are suitable for the compositions and methods described herein. Also, in U.S. Pat. No. 5,082,934, Saba et al describe a photoactivatible nucleoside analogue comprising a coumarin moiety linked through its phenyl ring to a ribose or deoxyribose sugar moiety without an intervening base moiety. In addition, U.S. patent application Ser. No. 10/735,174 discloses non-nucleosidic, stable, photoactive compounds that can be used as photo-crosslinking reagents in nucleic acid hybridization assays.

One advantage in the use of a composition comprising two oligonucleotides is that such a two oligonucleotide system can reduce the number of compositions which need to be attached to a solid support. For example, by providing one first oligonucleotide which is attached to a solid support which contains a universal tag, numerous second oligonucleotides can be designed which contain a common tag binding sequence, but which contain distinct target binding sequences.

In one embodiment, the second oligonucleotide can serve as a primer for a polymerase. The second oligonucleotide can hybridize to a target nucleic acid to form a hybrid complex. Where the target binding sequence of the second oligonucleotide is situated at the 3′ end, the oligonucleotide can be a primer for template-dependent synthesis by polymerase enzymes, for example by DNA polymerases and reverse transcriptases.

Method of Enriching for a Target Nucleic Acid

The present invention also provides a method for enriching a target nucleic acid. The method comprises the steps of providing a sample containing a nucleic acid; contacting the sample with a solid support/nucleic acid composition as described herein, under conditions that allow a hybrid complex (See, for example, FIG. 4A and FIG. 5A) to form between the target nucleic acid and the composition; and separating the hybrid complex from non-hybridized nucleic acids, whereby the target nucleic acid is enriched.

The composition useful for this method can be any composition which contains a target binding sequence, and encompasses compositions with one or more oligonucleotides.

The hybrid complex can be separated using a number of means known in the art, and is dependent upon the type of solid support used. In embodiments in which compositions comprising magnetic beads are employed, the hybrid complex, containing the solid support/nucleic acid composition described herein complexed with the target nucleic acid, can be separated by applying a magnetic field away from the rest of the sample. Alternatively, hybrid complexes can be separated using, for example, gravity sedimentation, filtration or centrifugation, depending on the physical property of the solid support used.

In one embodiment, the method further comprises the step of dissociating the hybrid complex after separating the hybrid complex from the rest of the sample (See, for example, FIG. 4B and FIG. 5B). The hybrid complex can be separated, for example, by heating the hybrid complex to above its melting temperature. In embodiments in which the composition does not comprise a second nucleotide, the hybrid complex is dissociated by heating the complex above its melting temperature, for example at least 5° C., 10° C., 15° C. or more above its melting temperature. In embodiments in which a second oligonucleotide comprising a target binding sequence and tag binding sequence is used, the hybrid complex between the composition and the target nucleic acid can be separated by applying a temperature that is between the melting temperature of the target:target binding sequence and that of the first oligonucleotide:second oligonucleotide complex (at the tag binding sequence). Alternatively, if the first and second oligonucleotide are joined by a crosslinking agent after they have formed a hybrid complex, the target nucleic acid can be dissociated from the composition at any temperature above its melting temperature, without regard to the melting temperature of the first:second oligonucleotide complex, since the latter complex is crosslinked. Finally, in embodiments in which an oligonucleotide is used which contains a cleavable linker between the tag binding sequence and target binding sequence, the hybrid complex can be dissociated by cleaving the linker.

It will be apparent to one of skill in the art that, after separating the hybrid complex, the hybrid complex can be further enriched by rinsing or washing the complex. After removing the remainder of the sample, the hybrid complex can be washed in solution containing mild detergents and/or chelating agents to remove residual non-target nucleic acids or any material present in the sample which may interfere with subsequent enzymatic steps, if any. While the washing solution can contain many additives, it is will be clear to one of skill in the art that the conditions of the wash should not dissociate the hybrid complex, and further that the target nucleic acid should not be damaged in anyway. In one embodiment, the washing step is performed at a temperature close to but below the melting temperature of the hybrid complex, for example 3° C., 5° C., 7° C., 10° C., 12° C. or more below the melting temperature of the hybrid complex.

It will be clear to one of skill in the art that, in embodiments in which a composition comprising a second oligonucleotide is used, it is not necessary that a hybrid complex be formed between the first oligonucleotide and second oligonucleotide prior to contacting with a sample. The two oligonucleotides can be added separately, and allowed to form a hybrid complex within the sample, simultaneous to, before or after the formation of the hybrid complex between the target binding sequence and the target nucleic acid.

As previously described, the compositions described herein also encompass compositions comprising target binding sequences for multiple target nucleic acids (See, for example, FIGS. 6-9). Multiple copies of a target binding sequence to the same target nucleic acid can be present on a single solid support (See, for example, FIGS. 6-8), either by attaching multiple copies of a first oligonucleotide to a single support (FIG. 7A), or by hybrid complex formation with second oligonucleotides, all containing the same target binding sequences (FIG. 7B). In addition, target bindings sequences to a plurality of different target nucleic acids can also be present on a single solid support (e.g., a bead), as is shown in FIG. 8, using only first oligonucleotides containing different target binding sequences (FIG. 8A), or by complex formation to second oligonucleotides containing different target binding sequences (FIG. 8B). Second oligonucleotides with different target binding sequences but with common tag binding sequences can be hybridized to first oligonucleotides, all attached to a support and all containing identical tags (FIG. 6).

The invention contemplates methods of enriching for a plurality of target nucleic acids (i.e., distinct sequences) by using compositions described herein. A plurality of target binding sequences can be provided using the compositions having a plurality of target binding sequences on single beads or a mixture solid supports each having one target binding sequence or multiple copies of the same target binding sequence. Examples of such compositions are shown in FIGS. 8 and 9.

Method of Performing Polymerase Reaction

In another aspect, the present invention provides a method of performing a polymerase reaction. The method comprises the following steps: providing a sample containing nucleic acids; contacting the sample with composition described herein under conditions that allow a hybrid complex to form between the nucleic acid and the composition; providing a polymerase and, extending the hybrid complex using the polymerase under conditions to allow a polymerase reaction.

In this aspect, the composition comprises a first oligonucleotide, attached to a solid support and wherein the first oligonucleotide does not serve as a primer for a polymerase enzyme; and a second oligonucleotide, comprising a tag binding sequence which is capable of hybridizing to the first oligonucleotide, and a target binding sequence which is capable of forming a hybrid complex with the target nucleic acid. While the first oligonucleotide is not capable of serving as a primer for polymerase, the second oligonucleotide can. In this aspect of the invention, the target binding sequence is located at the 3′ end of the second oligonucleotide, and, therefore, once it has formed a hybrid complex with the target nucleic acid, can serve as a primer for synthesis by a polymerase enzyme.

As described previously, the composition of the present invention affords the ability to enrich the target nucleic acid. Therefore, it will be apparent to one skilled in the art that the polymerase reaction using the composition of the invention can be performed after enriching the target nucleic acid. In one embodiment, the target nucleic acid is enriched prior to initiation of the polymerase reaction. This has the advantage that enrichment prior to polymerase reaction can serve to reduce the concentration of, or remove altogether, any contaminant present within the sample which might interfere with the action of the enzyme, or otherwise contribute to increased background in any way. In one embodiment, the enrichment step further comprises washing the hybrid complex. The complex can be washed as described previously.

The methods described herein also encompass methods of performing a polymerase reaction on a plurality of target nucleic acids. Hybrid complexes to a plurality of target nucleic acids can be formed using the compositions containing a plurality of target binding sequences as previously described (See, for example, FIG. 8 and FIG. 9).

The method contemplates the use of any polymerase known in the art, including DNA polymerases, RNA polymerases, reverse transcriptases, or mixtures of these enzymes.

In another embodiment, the target nucleic acid, including the polymerase reaction product, is enriched after completion of the polymerase reaction to a desired level. The complex can be washed to remove any material not part of the hybrid complex.

All publications, patents and published patent applications mentioned in the present specification, and references cited in said publications, are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A composition, comprising a first oligonucleotide attached to a solid support, wherein said oligonucleotide does not serve as a primer for a polymerase enzyme.

2. The composition of claim 1, wherein said first oligonucleotide comprises at least one locked nucleic acid.

3. The composition of claim 1, wherein said oligonucleotide is attached at its 3′ end to said solid support.

4. The composition of claim 1, wherein said solid support is paramagnetic.

5. The composition of claim 1, wherein said first oligonucleotide comprises natural nucleotides.

6. The composition of claim 1, wherein said first oligonucleotide comprises a non-natural nucleotide analog.

7. The composition of claim 1, wherein said first oligonucleotide is between 5 and 50 nucleotides in length, inclusive.

8. The composition of claim 1, further comprising a second oligonucleotide comprising a tag binding sequence which hybridizes to said first oligonucleotide.

9. The composition of claim 8, wherein said second oligonucleotide comprises at least one locked nucleic acid.

10. The composition of claim 9, wherein said tag binding sequence comprises at least one LNA.

11. The composition of claim 8, wherein said second oligonucleotide further comprises a target binding sequence, covalently linked to said tag binding sequence.

12. The composition of claim 10, wherein said target binding sequence is covalently linked at the 3′ end of said tag binding sequence.

13. The composition of claim 11, wherein said second oligonucleotide further comprises a cleavable linker, covalently linked between said tag binding sequence and said target binding sequence.

14. A method of enriching a target nucleic acid, comprising:
a. providing a sample containing nucleic acid;
b. contacting said sample with a composition of claim 1 under conditions that permit target nucleic acid in said sample to form a hybrid complex with said first oligonucleotide comprised by said composition; and
c. isolating said hybrid complex, whereby said target nucleic acid is enriched.

15. The method of claim 14, further comprising dissociating said hybrid complex after step (c).

16. The method of claim 14, wherein a plurality of different target nucleic acids is enriched.

17. A method of performing a polymerase reaction comprising:
a. providing a sample containing nucleic acids;
b. contacting said sample with a composition comprising a first oligonucleotide attached to a solid support, wherein said first oligonucleotide does not serve as a primer for a polymerase enzyme and said composition further comprises a second oligonucleotide comprising a tag binding sequence which hybridizes to said first oligonucleotide, said contacting performed under conditions that permit a hybrid complex to form between a said nucleic acid and said composition;
c. providing a polymerase; and,
d. extending said hybrid complex using said polymerase under conditions that permit nucleic acid polymerization.

18. The method of claim 17, further comprising enriching said hybrid complex after step (b).

19. The method of claim 17, further comprising enriching said hybrid complex after step (d).

20. The method of claim 17, wherein a plurality of different target nucleic acids is enriched.


SEQUENCE LISTING (5 sequences)

SEQ ID NO: 1 - DNA, 8 bp, Artificial Sequence
  oligo-dT example oligonucleotide
tttttttt

SEQ ID NO: 2 - DNA, 24 bp, Artificial Sequence
  Oligo dT, 24 Ts
tttttttttttttttttttttttt

SEQ ID NO: 3 - DNA, 24 bp, Artificial Sequence
  Example of target-specific oligonucleotide
  misc_feature (2)..(2): LNA analog of C
  misc_feature (6)..(6): LNA analog of C
  misc_feature (10)..(10): LNA analog of C
  misc_feature (13)..(13): LNA analog of C
  misc_feature (15)..(15): LNA analog of G
  misc_feature (19)..(19): LNA analog of C
  misc_feature (22)..(22): LNA analog of C
acaggcagccagctgttcctcctt

SEQ ID NO: 4 - DNA, 56 bp, Artificial Sequence
  Further example of target binding oligonucleotide
  misc_feature (1)..(1): LNA analog of A
  misc_feature (3)..(3): LNA analog of A
  misc_feature (5)..(5): LNA analog of A
  misc_feature (7)..(7): LNA analog of A
  misc_feature (9)..(9): LNA analog of A
  misc_feature (11)..(11): LNA analog of A
  misc_feature (13)..(13): LNA analog of A
  misc_feature (15)..(15): LNA analog of A
  misc_feature (17)..(17): LNA analog of A
  misc_feature (19)..(19): LNA analog of A
  misc_feature (21)..(21): LNA analog of A
  misc_feature (23)..(23): LNA analog of A
aaaaaaaaaaaaaaaaaaaaaaaagcggccgcacaggcagccagctgttcctcctt

SEQ ID NO: 5 - DNA, 9 bp, Artificial Sequence
  Oligo dA example oligonucleotide
  misc_feature (2)..(2): LNA analog of A
  misc_feature (4)..(4): LNA analog of A
  misc_feature (6)..(6): LNA analog of A
  misc_feature (8)..(8): LNA analog of A
aaaaaaaaa